(19) United States
(12) Patent Application Publication
(10) Pub. No.: US 2024/0100266 A1
(43) Pub. Date: Mar. 28, 2024

(54) SURGICAL TOOL FOR TRANSRETINAL PNEUMATIC DISPLACEMENT OF SUB-RETINAL HEMORRHAGE

(71) Applicant: OPTICGON, LLC., Flushing, NY (US)

(72) Inventor: Ian H. CHAN, Flushing, NY (US)

(21) Appl. No.: 18/374,304

(22) Filed: Sep. 28, 2023

Related U.S. Application Data
(60) Provisional application No. 63/410,946, filed on Sep. 28, 2022.

(51) Int. Cl. A61M 13/00; A61F 9/007; A61K 31/573; A61K 38/17; A61K 39/395

(52) U.S. Cl. CPC A61M 13/003; A61F 9/007; A61K 31/573; A61K 38/179; A61K 39/3955; A61B 2017/32035; A61M 2205/3344; A61M 2210/0612

(57) ABSTRACT

This disclosure provides an ophthalmological surgical tool to perform transretinal pneumatic displacement of sub-retinal hemorrhage, and uses thereof. The surgical tool comprises a gas cannula which is configured to dispense air or medical gas in an air-knife pattern to displace or disrupt sub-retinal hemorrhage away from the macula.

### CROSS-REFERENCE TO RELATED APPLICATIONS

This application claims priority to U.S. Provisional Application No. 63/410,946, filed on Sep. 28, 2022, the contents of which are herein incorporated in their entirety.

### FIELD

This disclosure relates to the field of retinal surgical instrument for treating sub-retinal hemorrhage. In particular, this disclosure relates to an air cannula configured to pneumatically displace and/or disrupt sub-retinal hemorrhages. This invention furthermore relates to a method of displacing or disrupting a sub-retinal hemorrhage by applying pressurized air or medical grade gas to the hemorrhage using a gas cannula as described herein to pneumatically displace or disrupt the sub-retinal hemorrhage away from the retina.

### BACKGROUND

Sub-retinal hemorrhage caused by age-related macular degeneration or macroaneurysms has been associated with a poor visual outcome. Treatments for sub-retinal hemorrhage include vitreous surgery but have resulted in inconsistent results. Improvements to vitreous surgery include the use of a small retinotomy, tissue-type plasminogen activator (tPA), and perfluorocarbon liquid, but even with such improvements, surgical methods have not been ideal.

Some methods of surgical treatment for sub-retinal hemorrhage from exudative age-related macular degeneration, polypoidal choroidal vasculopathy, high myopia, and other causes of choroidal neovascularization include pneumatic displacement by injecting gas directly into the vitreous cavity followed by face down head positioning. Other methods based on pneumatic displacement involve sub-retinal gas injection by introducing a needle through the retina into the sub-retinal space with or without tissue plasminogen. Previous concepts in clearing submacular hemorrhage require injection of gas with or without tissue plasminogen into the sub-retinal space or injection into the vitreous cavity.

### SUMMARY

In one aspect, this disclosure relates to a surgical system used to perform transretinal pneumatic displacement of a sub-retinal hemorrhage. The surgical system comprises a gas cannula configured to be inserted into the eye of a subject via a trocar, and wherein the distal end of the cannula is placed intraocularly at close proximity to the retina. The gas cannula can provide pressurized medical grade gas or filtered air to displace or disrupt a sub-retinal hemorrhage away from the macula in a transretinal approach. The distal end of the gas cannula comprises a tip of the gas cannula may be round or spatulated to create an air-knife, or a plane of stream of gas.

In one aspect, the pressure delivered to the distal tip of the gas cannula can be modulated. In some aspects, the gas pressure can be modulated by a gas pressure modulator handpiece.

In one aspect, this disclosure provides for a gas pressure modulator handpiece comprising: a wall surrounding an open lumen; a longitudinal axis along the open lumen; a proximal end; and a distal end, where the distal end comprises one or more openings configured to allow for pressurized air or medical grade gas to propagate and be ejected through the distal end, the proximal end is in fluidic communication with a filtered gas feed tube, and the one or more openings comprise a profile configured to propagate and eject dispensed air from said distal end in a laminar flow pattern. In some aspects, the distal end of the gas pressure modulator handpiece can be a gas cannula as described herein.

In some aspects, the laminar flow pattern can operate as an air knife.

In some aspects, the open lumen can comprise a longitudinal diameter ranging from 14-31 gauge. In some aspects, the propagated and ejected gas can be propagated in a continuous or pulsed manner. In some aspects, the one or more distal openings can independently comprise a cross-sectional profile selected from a spatulated, fan-shaped, round, prolate round, or rectangular shape. In some aspects, the one or more distal openings can comprise a prolate round shape.

In some aspects, the gas pressure modulator handpiece can further comprise an inline gas filter, where the filtered gas feed tube is in fluidic communication with an inline gas filter. In some aspects, the gas pressure modulator handpiece can further comprise an inline gas filter, where the inline gas filter is in fluidic communication with a pre-filtered gas feed tube. In some aspects, the gas pressure modulator handpiece can further comprise a gas regulator, where the pre-filtered gas feed tube is in fluidic communication with a gas regulator. In some aspects, the gas pressure modulator handpiece can further comprise a pressurized gas source where the gas regulator is in fluidic communication with a pressurized gas source. In some aspects, the gas pressure modulator handpiece can further comprise a gas cylinder where the pressurized gas source is a pressurized gas cylinder.

In some aspects, the wall surrounding an open lumen in the gas pressure modulator handpiece can comprise one or a plurality of first gas egress ports. The number of first gas egress ports can be from 1 to 1000. Each of the first gas egress ports can be independently selected from a mesh grid, a hole, a wireframe grid, or a porous frit.

In some aspects, the gas pressure modulator handpiece can further comprise a slide rack comprising a slider button, a longitudinally moveable tube, and a modulated exit gas tube, where the slider button and slide rack are configured to longitudinally move to selectively cover the one or a plurality of first gas egress ports. In some aspects, the gas pressure modulator handpiece can further comprise a guard membrane, where the guard membrane comprises one or a plurality of second gas egress ports. The number of second gas egress ports can be from 1 to 1000. Each of the second gas egress ports can independently be selected from a mesh grid, a hole, a wireframe grid, or a porous frit.

In some aspects, the pressure of air directed through the modulated exit gas tube when pressurized air is presented to the gas pressure modular handpiece through the proximal end decreases when the slider button and slide rack are not covering the one or a plurality of first gas egress ports, relative to when the slider button and slide rack are covering the one or a plurality of first gas egress ports. In this manner, the gas pressure, and gas flow, can be controlled by the hand of an operator in a mechanical manner without the need to an electronic controller unit and/or a controller unit out of visual range of the operator.

In some aspects, the gas pressure modulator handpiece comprises a longitudinal axis which can be straight, curved, or comprise two or more longitudinal axes such that the modulated exit gas tube is bent.

In some aspects, this disclosure provides for a surgical system, comprising: a gas pressure modulator handpiece as described herein, a gas regulator; and a pressurized gas cylinder, where the handpiece is configured to selectively control the flow rate of the pressurized gas into the modulated exit gas tube. In some aspects, the gas pressure modulator handpiece comprises a wall surrounding an open lumen; a longitudinal axis along the open lumen; a proximal end; and a distal end, where the distal end comprises one or more openings configured to allow for pressurized air or medical grade gas to propagate and be ejected through the distal end, the proximal end is in fluidic communication with a filtered gas feed tube, and the one or more openings comprise a profile configured to propagate and eject dispensed air from said distal end in a laminar flow pattern. In some aspects, the gas pressure modulator handpiece can further comprise an inline gas filter, where the filtered gas feed tube is in fluidic communication with an inline gas filter. In some aspects, the gas pressure modulator handpiece can further comprise an inline gas filter, where the inline gas filter is in fluidic communication with a pre-filtered gas feed tube. In some aspects, the gas pressure modulator handpiece can further comprise a gas regulator, where the pre-filtered gas feed tube is in fluidic communication with a gas regulator. In some aspects, the gas pressure modulator handpiece can further comprise a pressurized gas source where the gas regulator is in fluidic communication with a pressurized gas source. In some aspects, the gas pressure modulator handpiece can further comprise a gas cylinder where the pressurized gas source is a pressurized gas cylinder.

In some aspects, the flow rate of the pressurized gas emitted through the distal opening can range from 0.1 to 20 standard liters per minute (SLPM). In some aspects, the pressurized gas emitted through the distal opening can be 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20 SLPM or any value between the aforementioned values. In some aspects, the pressure of the pressurized gas emitted through the distal opening can range from 0.1 to 30 psi. In some aspects, the pressure of the pressurized gas emitted through the distal opening can be 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30 psi, or any value between the aforementioned values. In some aspects, the pressurized gas is selected from medical-grade gas or air.

In some aspects, this disclosure provides for a method of disrupting and/or dispersing a sub-retinal hemorrhage in the eye of a subject in need thereof, the method comprising: configuring the distal end of a gas pressure modulator handpiece as described herein to be proximate to a sub-retinal hemorrhage in the eye of a subject; presenting a pressurized gas to the sub-retinal hemorrhage through the gas pressure modulator handpiece; and moving the distal end of said gas pressure modulator handpiece in a transretinal manner to disrupt or disperse the sub-retinal hemorrhage. In some aspects, the gas cannula is introduced into the eye through a trocar. In some aspects, the gas pressure modulator handpiece comprises a wall surrounding an open lumen; a longitudinal axis along the open lumen; a proximal end; and a distal end, where the distal end comprises one or more openings configured to allow for pressurized air or medical grade gas to propagate and be ejected through the distal end, the proximal end is in fluidic communication with a filtered gas feed tube, and the one or more openings comprise a profile configured to propagate and eject dispensed air from said distal end in a laminar flow pattern. In some aspects, the gas pressure modulator handpiece can further comprise an inline gas filter, where the filtered gas feed tube is in fluidic communication with an inline gas filter. In some aspects, the gas pressure modulator handpiece can further comprise an inline gas filter, where the inline gas filter is in fluidic communication with a pre-filtered gas feed tube. In some aspects, the gas pressure modulator handpiece can further comprise a gas regulator, where the pre-filtered gas feed tube is in fluidic communication with a gas regulator. In some aspects, the gas pressure modulator handpiece can further comprise a pressurized gas source where the gas regulator is in fluidic communication with a pressurized gas source. In some aspects, the gas pressure modulator handpiece can further comprise a gas cylinder where the pressurized gas source is a pressurized gas cylinder.

In one aspect, this disclosure provides for a surgical system which comprises a handpiece with a switch to control the gas flow, and a gas cannula described herein. In some aspects, the gas flow can be controlled by a foot pedal connected to the gas pressure controller. The surgical system comprises a gas pressure controller which comprises a direct connection to a pressured medical gas tank and/or an air compressor. The gas can be medical-grade air, optionally filtered. The gas pressure controller can generate a continuous stream of gas flow, pulses or sinusoidal waves of gas flow to aid in disbursement and disintegration of sub-retinal blood clots and displacement of the sub-retinal hemorrhage away from the macular region.

In some aspects, this disclosure provides for a gas cannula surrounding an open lumen, comprising a proximal end and a distal end, where the distal end comprises one or more openings configured to allow for pressurized air or medical grade gas to propagate and be ejected through, and where the distal end is configured to be spatulated such that the propagated and ejected air is dispensed from said distal end in a laminar flow pattern.

In some aspects, the distal end of the gas cannula comprises a rectangular opening. In some aspects, the distal end comprises a prolate round opening. In some embodiments, the prolate round opening on the distal end is along the horizontal axis of the gas cannula. The proximal end can be connected to a pressurized gas source. The pressurized gas source can be a medical grade gas cylinder or an air compressor. The pressurized air source can further comprise an air filter.

In some aspects, the laminar flow pattern can be in the form of an air knife. In some aspects, the laminar flow can be propagated in a continuous or pulsed manner. In some aspects, the laminar flow can be propagated a in wave pattern. The wave mode can be in a sinusoidal wave pattern. The sinusoidal wave pattern can be of a selected range of frequencies and amplitudes. The pulse pattern can be of a selected range of frequencies and amplitudes.

In some aspects, the one or more distal openings independently can comprise a cross-sectional profile selected from a spatulated, regtangular, or fan-shaped opening to create an air knife. In some aspects, the one or more distal openings can comprise a round opening.

In some aspects, the open lumen can comprise a cross-sectional longitudinal diameter ranging from 14-31 gauge, from 18 to 24 gauge, from 20 to 22 gauge, or any gauge between any of the aforementioned ranges. In some aspects, the open lumen can comprise a cross-sectional longitudinal diameter selected from 14, 15, 16, 17, 18, 19, 20, 21, 22, 23, 24, 25, 26, 27, 28, 29, 30, or 31 gauge. In some embodiments, the longitudinal diameter ranges from 0.3 to 1.6 mm.

In some aspects, the distal end of the gas cannula can comprise a tip. In some aspects, the tip can be made of silicone, polyethylene, polypropylene, polycarbonate, or polytetrafluoroethylene.

The gas cannula can comprise a longitudinal axis which can be curved. The gas cannula can comprise two or more longitudinal axes such that the gas cannula is angled along two or more planes. The gas cannula can comprise two longitudinal axes which can be angled at an angle of about 135 to about 165 degrees in relation to each axis.

In some aspects, this disclosure provides for a surgical system, comprising: a gas cannula as described herein; a handpiece coupled to a pressured gas source, where the handpiece is configured to selectively control the fluidic contact of pressured air or medical gas into the open lumen of the gas cannula from the pressured gas source, and where the handpiece is connected to the gas cannula; and a gas source controller connected to the proximal end of the gas cannula, wherein the gas source controller delivers pressured gas to the handpiece.

In some aspects, the handpiece can comprise a toggle switch to control the flow of gas through the lumen of the handpiece. The toggle switch can be coupled to a valve which modulates the flow of gas. The valve can be binary to control whether the gas flow or not, or the valve can be modulated to control the gas pressure of the gas.

The gas flow can be controlled by a switch on the foot pedal or by a switch on the handpiece. In some aspects, the handpiece can further comprise a sensor configured to be located at the distal end of the gas line to monitor the gas flow and gas pressure. In some aspects, the surgical system can further comprise a foot pedal which is configured to control the gas pressure. The gas source controller can control the gas pressure in a linear fashion based on the pressure applied to the foot pedal.

In some aspects, the surgical system further comprises a computer system comprising a computer monitor and a dial or haptic feedback display which can be configured to control the gas pressure.

In some aspects, the gas pressure can range from 0.1 to 100 mm Hg, from 1 to 80 mm Hg, from 5 to 20 mm Hg, from 10 to 18 mm Hg, or from 12 to 16 mm Hg, or any pressure between any of the aforementioned ranges. In some aspects, the gas pressure can range from 10 to 90 mm Hg, from 20 to 80 mm Hg, from 30 to 70 mm Hg, from 30 to 50 mm Hg, from 35 to 45 mm Hg, or from 37 to 42 mm Hg, or any pressure between any of the aforementioned ranges. In some aspects, the gas pressure can be about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 ,25, 26, 27, 28, 29 ,30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mm Hg or any pressure between any of the aforementioned values.

In some aspects, the surgical system can further comprise a gas compressor and a medical-grade air filter to produce the medical-grade gas or air at a selected gas pressure.

In some aspects, the surgical system can further comprise a valved or regulated connection to a pressurized medical gas tank. The valve or regulator can present the medical-grade gas or air to the handpiece at a selected gas pressure.

In some aspects, this disclosure provides for a method of disrupting and/or dispersing a sub-retinal hemorrhage in the eye of a subject in need thereof, the method comprising: contacting a sub-retinal hemorrhage with a gas cannula as described herein; presenting a gas flow to the sub-retinal hemorrhage through the gas cannula; and moving the gas cannula in a transretinal manner to disrupt or disperse the sub-retinal hemorrhage. In some aspects, the gas cannula can be introduced into the eye through a trocar.

In some aspects, this disclosure provides for a kit comprising the gas cannula as described herein and a therapeutic agent. The therapeutic agent can be selected from an anti-VEGF agent or a steroid. The anti-VEGF agent can be selected from: Avastin (bevacizumab), Lucentis (ranibizumab), Eylea (aflibercept), Beovu (brolucizumab), and Vabysmo (faricimab). The steroid can be selected from: Triesence (triamcinolone acetonide), and Kenalog (triamcinolone acetonide).

### BRIEF DESCRIPTION OF THE DRAWINGS

The drawings form part of the present specification and are included to further demonstrate certain aspects of the embodiments described herein. These embodiments may be better understood by reference to one or more of the following drawings in combination with the detailed description.

FIG. 1 is a perspective view of a representative gas cannula 100 of the disclosure comprising a continuous wall (not identified) surrounding an open lumen 102. The tip of the gas cannula 106 comprises two longitudinal axes such that the lumen is bent, and further comprises a prolate round opening 108 where pressurized gas will be emitted.

FIG. 2 is a perspective view of a representative gas cannula of the disclosure wherein the tip of the gas cannula where the cannula tip is straight and further comprises a round opening 202.

FIG. 3 is a perspective view of a representative gas cannula of the disclosure wherein the tip of the gas cannula where the cannula tip is angled and spatulated and further comprising a rectangular opening 301.

FIG. 4 is perspective view of a representative gas cannula handpiece of the disclosure comprising a handpiece 401 which is connected to the gas cannula at the distal end and a soft gas tubing on the proximal end, and further comprises a press switch 402 which modulates the flow of the pressured gas to the cannula 403.

FIG. 5A is a side view of a representative gas cannula of the disclosure wherein the open lumen 102 is surrounded by a continuous wall 101 and the path of the lumen comprises two longitudinal axes such that the lumen is bent, and further comprising an opening 108.

FIG. 5B is a side view of a representative gas cannula of the disclosure wherein the open lumen 102 is surrounded by a continuous wall 101 and the path of the lumen comprises a curved axis such that the lumen is curved.

FIG. 6 is a schematic diagram of a representative surgical system of this disclosure comprising a gas controller unit 601 which generates pressurized medical gas or filtered air at a selected pressure/flow rate to be delivered to a handpiece. In some embodiments, the pressurized filter air is provided by an internal air compressor configured to be positioned within the gas controller 601. In some embodiments, the pressurized filter is provided by a gas cylinder 603. The surgical system further comprises a handpiece 602 where the proximal end is connected to the gas controller unit with gas tubing and the distal end is connected to the gas cannula 603 to be inserted into the eye of a subject. In some embodiments, the surgical system further comprises a foot pedal 604 which is in electronic communication with the gas controller unit and modulates in a linear fashion the air flow from the compressed air source based on pedal depression.

FIG. 7 shows a microscopy image of the eyeball with the blood clot outlined.

FIG. 8 shows a microscopy image of the eyeball after performing this procedure, where the outline of the blood clot is significantly reduced in area.

FIG. 9 depicts a representative embodiment of a surgical system of this disclosure which includes a gas pressure modulator handpiece 905, a gas regulator 902, a pressurized gas cylinder 901, a pre-filtered gas feed tube 903, an inline gas filter 904, a filtered gas feed tube 909, a guard membrane 906, a slider button 907, and a modulated gas tube 908.

FIG. 10 depicts a perspective view of a representative embodiment of a gas pressure modulator handpiece 905 of this disclosure, including an inline gas filter 904, a filtered gas feed tube 909, a slider button 907, a modulated gas tube 908, a distal end 910, a longitudinally movable tube 911, and an outer handpiece wall 912.

FIG. 11 depicts a perspective view of a representative embodiment of a gas pressure modulator handpiece 905 of this disclosure, including a distal opening 915, wherein the distal opening 915 comprises a prolate round shape.

FIG. 12 depicts a perspective view of a representative embodiment of a gas pressure modulator handpiece 905 of this disclosure, including a slide rack 914 relative to a slider button 907 and longitudinally movable tube 911. In some embodiments, the slider button 907 and longitudinally movable tube 911 are made of the same construction.

FIG. 13 depicts a perspective view of a representative embodiment of a gas pressure modulator handpiece 905 of this disclosure, including a guard membrane 906 affixed to a outer handpiece wall 912. In some embodiments, the guard membrane 906 is connected to the outer handpiece wall 912 by being of the same construction as the outer handpiece wall 912, being glued or hot-melted to the outer handpiece wall 912, or being clipped to the outer handpiece wall 912. The gas pressure modulator handpiece can include a plurality of first gas egress ports 916a, 916b, 916c, and 916d which allow for gas to be partially released from the gas pressure modulator handpiece when the longitudinally movable tube 911 covers the one or more first gas egress ports. For example, when the longitudinally movable tube 911 is configured to cover two of the first egress ports 916c and 916d, a portion of the pressurized gas introduced into the gas pressure modulator handpiece from the filtered gas feed tube 909 can traverse through the uncovered first gas egress ports 916a and 916b to reduce the portion of gas leaving the gas modulator handpiece through the modulated exit gas tube 913. When the longitudinally movable tube 911 is configured to not cover any of the first egress ports, a lower portion of the pressurized gas introduced into the gas pressure modulator handpiece from the filtered gas feed tube 909 can escape through the one or a plurality of first gas egress ports such that a lower portion of gas leaves the gas modulator handpiece through the modulated exit gas tube 913, resulting in a lower pressure presented to the distal end of the gas pressure modulator handpiece 910. When the longitudinally movable tube 911 is configured to cover all of the first egress ports 916a, 916b, 916c, and 916d, the gas introduced into the gas pressure modulator handpiece is retained in the gas pressure modulator resulting in a higher pressure which is presented to the distal end of the gas pressure modulator handpiece 910. Thus, selective configuring of the longitudinally movable tube 911 relative to the one or plurality of first gas egress ports can modulate the gas pressure presented at the distal end of the gas pressure modulator handpiece 910 in a mechanically controllable fashion. Also depicted is a guard membrane 906 comprising second gas egress ports 917a, 917b, 917c, and 917d which allow for gas traversing through the one or plurality of first gas egress ports 916a, 916b, 916c, and 916d to escape from the guard membrane. The position of the one or plurality of second gas egress ports can be configured to be placed at a selected distance from the one or plurality of first gas egress ports such that no external particles can directly enter the gas pressure modulator handpiece through the first gas egress ports.

FIG. 14 depicts a reduced perspective view of a representative embodiment of a gas pressure modulator handpiece 905 of this disclosure, including a filtered gas feed tube (not indicated) and a modulated exit gas tube (not indicated).

FIG. 15 depicts a cross-section profile view of a representative embodiment of a gas pressure modulator handpiece 905 of this disclosure, including a second gas egress port 917, first gas egress port 916, guard membrane 906, longitudinally movable tube 911 which surrounds a modulated exit gas tube 913, a slide rack 914, a slider button 907, an outer handpiece wall 912, a male clip 919 affixed to the outer handpiece wall, a female clip 918 affixed to the guard membrane 906, wherein the male clip 919 and female clip 918 are mated to connect the guard membrane 906 to the gas modulator handpiece 905.

FIG. 16 depicts a cross-section side view of a modulated gas tube 908 which tapers to a distal end 910 and further comprises a distal opening 915. The taper can occur along one dimension (e.g., top-to-bottom in the depicted image) such that the shape of the distal opening 915 is flat, resulting in lamellar flow of gas when exiting the distal opening.

FIG. 17 depicts a side profile view of a representative embodiment of a gas pressure modulator handpiece of this disclosure in a gas egress port open configuration resulting in a lower pressure delivered to the modulated gas tube 908. The longitudinally movable tube 911 is configured to open (or unblock) the first gas egress port 906 by the operator pushing on the slider button 907 in a direction about parallel to the longitudinally movable tube 911. Gas flow (dark arrows) presented to the gas pressure modulator handpiece from the filtered gas feed tube 909 is split between exiting though the first gas egress port 906 and the modulated exit gas tube 913 and then to the modulated gas tube 908.

FIG. 18 depicts a side profile view of a representative embodiment of a gas pressure modulator handpiece of this disclosure in a gas egress port closed configuration resulting in a higher pressure delivered to the modulated gas tube 908. The longitudinally movable tube 911 is configured to close (or block) the first gas egress port 906 by the operator pushing on the slider button 907 in a direction about parallel to the longitudinally movable tube 911. Gas flow (dark arrows) presented to the gas pressure modulator handpiece from the filtered gas feed tube 909 are directed primarily to the modulated exit gas tube 913 and then to the modulated gas tube 908, resulting in a higher output pressure from the gas pressure modulator handpiece than by the configuration presented in FIG. 17.

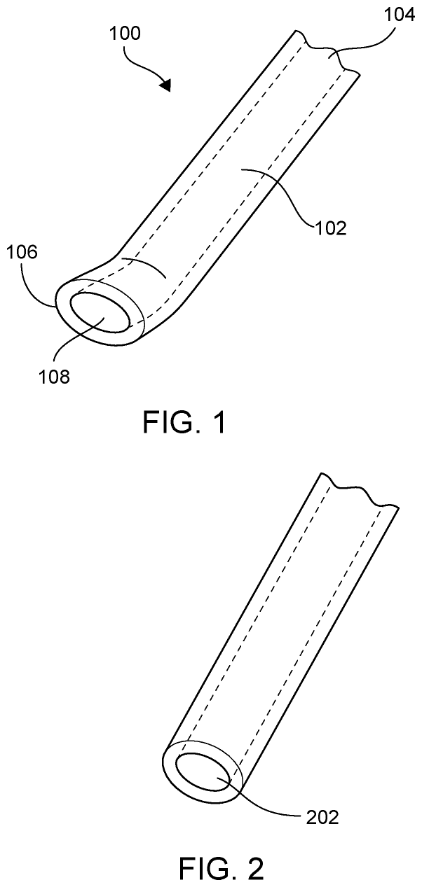

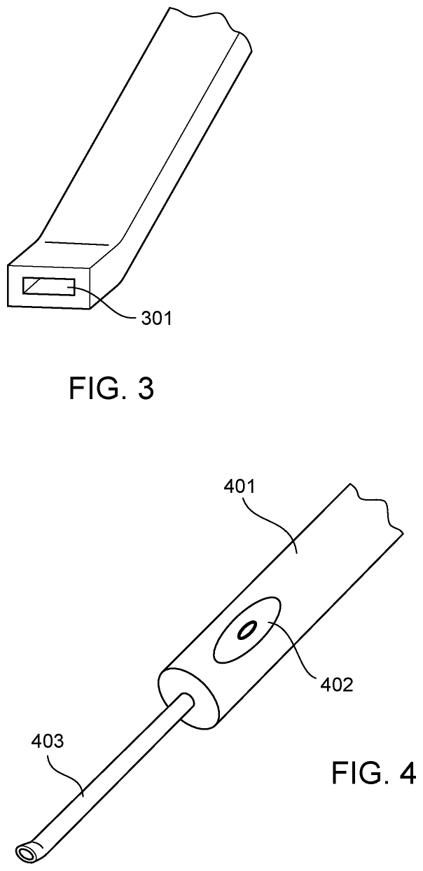

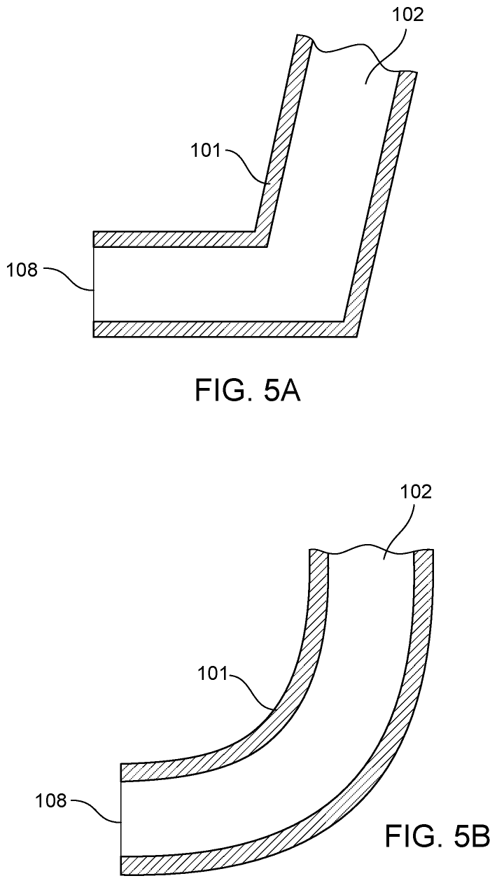

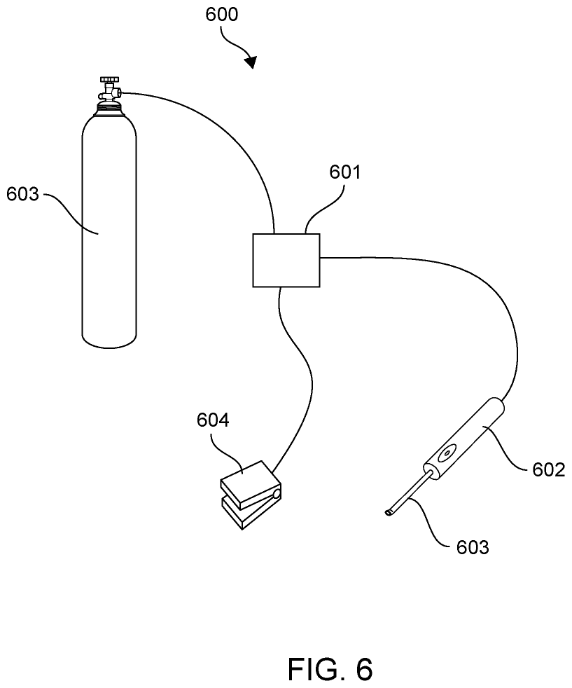

### DETAILED DESCRIPTION

Certain Definitions

Unless defined otherwise, all technical and scientific terms used herein have the same meaning as commonly understood by one of ordinary skill in the art to which this disclosure is related.

Units, prefixes, and symbols are denoted in their System International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “about” means±10%.

An “air knife” is a cleaning, drying and/or cooling device used in manufacturing and other processes. By creating a high intensity, balanced sheet of laminar airflow across the entire length of a selected area. Laminar airflow refers to a uniform layer airflow that does not mix with the other layers nearby. The process of air knifing can comprise applying at least one powerful jet of a gas (air or medical grade gas) to apply pressure to a selected area to move coagulated blood or excess ocular fluid from a selected area. The air flow of the air knife can be applied in a direction that is generally transverse, such as orthogonal, relative to the longitudinal axis of the device. In some embodiments, the air knife is applied to the retina of an eye in a transretinal manner to disrupt or disperse a sub-retinal hemorrhage.

A “handpiece” is the part of a mechanized device designed to be held, affixed to, or manipulated by a human hand. In some embodiments, the handpiece can comprise a grip portion. In some embodiments, the handpiece can comprise a strap which affixes the handpiece to a human hand. In some embodiments, the handpiece comprises an air tube which is connected to a gas source as described herein. In some embodiments, the handpiece comprises a toggle switch, as described herein. In some embodiments, the handpiece comprises a lumen, as described herein. In some embodiments, the handpiece comprises a switch which is in (either pneumatic or electronic) communication with a binary valve to selectively control whether gas is emitted through the cannula while a foot pedal controls the gas pressure as a function of the compression applied to said foot pedal. In some embodiments, the handpiece comprises a longitudinally movable tube, one or a plurality of first gas egress ports, and a modulated exit gas tube such that when the longitudinally movable tube blocks the gas egress ports, the pressure delivered to the modulated exit gas tube is higher than when the longitudinally movable tube does not block the gas egress ports.

As used herein, the term “administering” refers to the physical introduction of an agent to a subject, using any of the various methods and delivery systems known to those skilled in the art. Exemplary routes of administration for the methods disclosed herein include trans-retinal, intra-ocular, intravitreal, oral, transdermal, transmuscular, sublingual, intraperitonal, or by suppository.

As used herein, the term “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, the term “subject” as used herein refers to humans, higher non-human primates, rodents, rabbits, horses, pigs, sheep, dogs and cats. In one embodiment, the subject is a human.

As used herein, the terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and increasing visual acuity or ability of a subject, whether detectable or undetectable.

As described herein, any concentration range, percentage range, ratio range or integer range is to be understood to include the value of any integer within the recited range and, when appropriate, fractions thereof (including one-tenth and one-hundredth of an integer), unless otherwise indicated.

### Gas Cannula

In some embodiments, this disclosure relates to a surgical device for the transretinal pneumatic displacement and/or disruption of a hemorrhage (including a sub-retinal hemorrhage) in which a gas cannula comprising a nozzle at the distal end has a configuration selected to deliver an air knife pneumatic pressure. The nozzle is placed directly over a retina which presents a sub-retinal hemorrhage. The surgical device can be angled at the sub-retinal hemorrhage at a sufficient angle to displace or disrupt away the sub-retinal hemorrhage from the macular region. The flow pattern from the nozzle can be shaped to be such that the air flow acts as an air knife.

The gas cannulas described herein reduces the risk of creating a retinal break or a retinal detachment which could otherwise occur during a sub-retinal injection method for treating a sub-retinal hemorrhage. The gas cannulas described herein also improve upon the efficacy of pneumatic displacement by intravitreal injection because in conventional pneumatic displacement by intravitreal injection, the gas pressure applied toward the macular region is limited, thereby reducing the efficacy of this method. Transretinal pneumatic displacement offers a way to directly apply the gas pressure over the sub-retinal hemorrhage which improves the direct impact and efficacy of this approach. Retina damage is also minimized as the pneumatic pressure is transferred from the retinal tissue to the underlying hemorrhage which reduces impact of pressure-induced retinal damage.

The gas cannula comprises a continuous wall surrounding an open lumen. The interior surface of the open lumen is intended to be smooth to present pressurized gas flow in a laminar fashion. The gas cannula can be of a straight, bent, or curved shape, characterized in having one axis or a plurality of axes. In some embodiments, the lumen comprises two axes such that the gas cannula is bent. In some embodiments, the lumen comprises a curved axis such that the gas cannula is curved.

The gas cannula further comprises a proximal end and a distal end. The distal end comprises one or more openings configured to allow for pressurized air or medical grade gas to propagate and be ejected from said open lumen to said distal end in a laminar flow pattern. The proximal end can be configured to be mated to a handpiece which can valvably control the gas pressure flow in a binary manner. In some embodiments, the laminar flow pattern is in the form of an air knife.

In some embodiments, the open lumen has an internal diameter ranging from 10-34 gauge (0.051 mm ID to 2.7 mm ID). In some embodiments, the internal diameter is the longitudinal diameter.

The one or more distal openings independently comprise a cross-sectional profile selected from a spatulated, fan-shaped, round, prolate round, or rectangular shape, or any shape which can dispense pressurized gas in an air knife flow pattern.

In some embodiments, the gas cannula can be made of a soft plastic. The soft plastic can be made of a polypropylene, polyethylene, teflon, or silicone material. In some embodiments, the gas cannula can comprise a tip. The tip can be made of silicone, and the gas cannula can be made from another type of soft plastic material as described herein.

### Gas Pressure Modulator Handpiece

In some embodiments, this disclosure provides for a gas pressure modulator handpiece. The gas pressure modulator handpiece can modulate the pressure of gas presented the eye of a subject by mechanically moving a component of the handpiece such that there is no need for an external computer control box, and foot pedal, to control the gas pressure. One advantage of controlling the pressure mechanically with the handpiece is that the operator (the surgeon) does not need to change their field of view when modulating the gas pressure. By setting the pressure from the gas tank via a gas regulator to 30 psi or less, the handpiece can modulate the pressure down from 30 psi or less to the appropriate level for the transretinal pneumatic displacement of sub-retinal hemorrhage procedure.

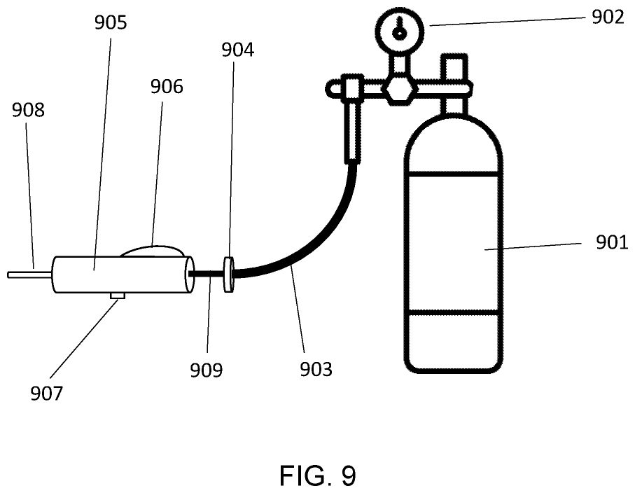

As depicted in FIG. 9, a system comprising the gas pressure modulator handpiece 905 only includes mechanical components, and no electronic devices are required. Regulatory requirements are also reduced with the absence of electronic devices within the system.

Also as depicted in FIG. 9, gas pressure is initially controlled by a gas regulator 902, then traverses through a pre-filtered gas feed tube 903 before contacting an inline gas filter 904. In some embodiments, the inline gas filter can be a frit, a membrane, or a mesh. In some embodiments, the inline gas filter can be a Pall Oxygenator Gas Filter (Pall Corp.).

After contacting the inline gas filter 904, the filtered gas traverses through a filter gas feed tube 909 before entering the gas pressure modulator handpiece 905.

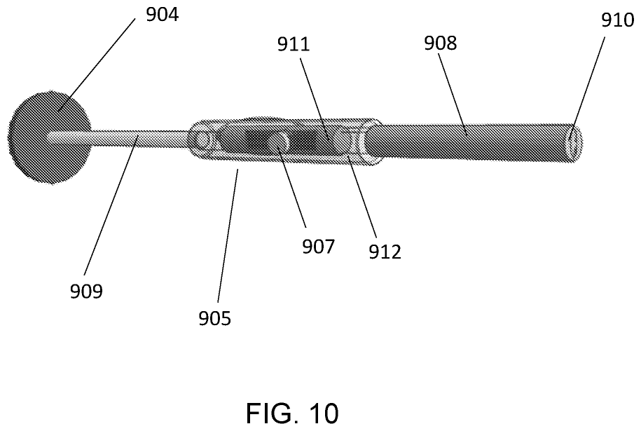

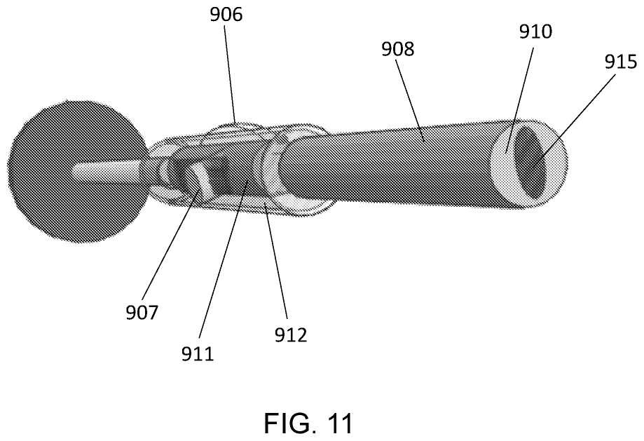

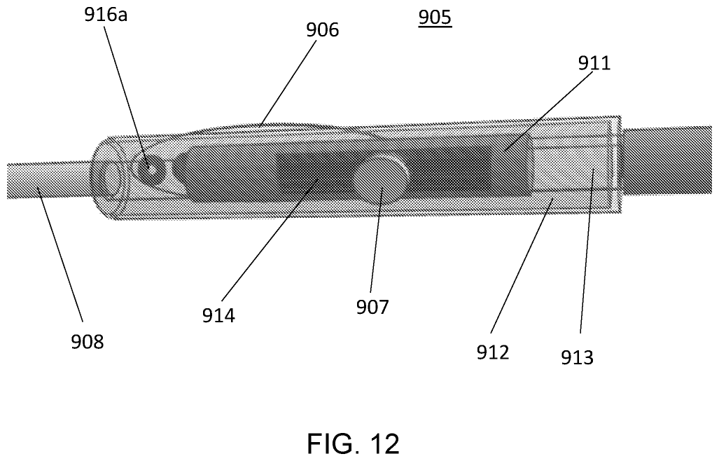

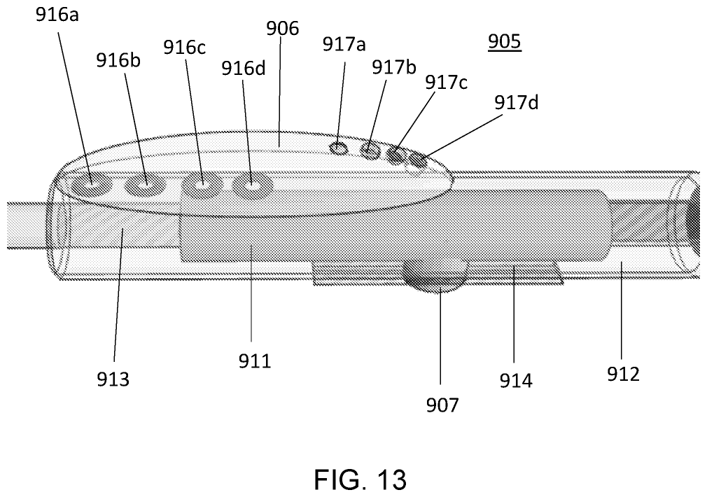

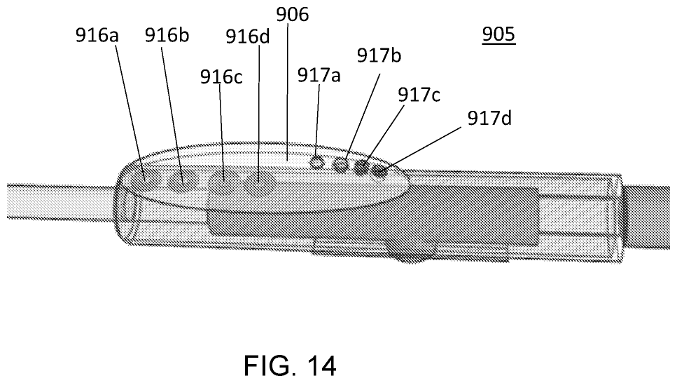

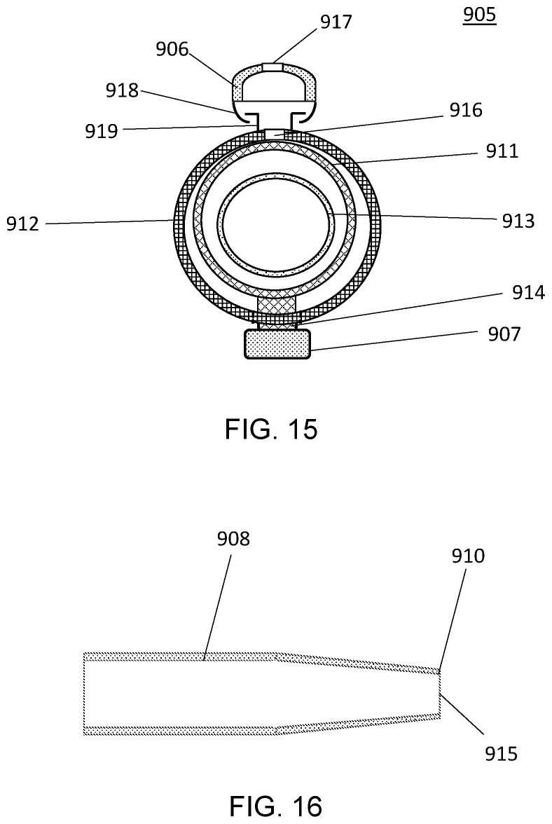

As depicted in FIG. 10, the filtered gas entering the gas pressure modulator handpiece 905 traverses a longitudinally movable tube 911 within an outer handpiece wall 912 and then through a modulated exit gas tube 913 (depicted in FIG. 13) and then through a modulated gas tube 908 before exiting through a distal end 910. In some embodiments, the modulated gas tube 908 is a cannula, having the properties of a cannula as described herein.

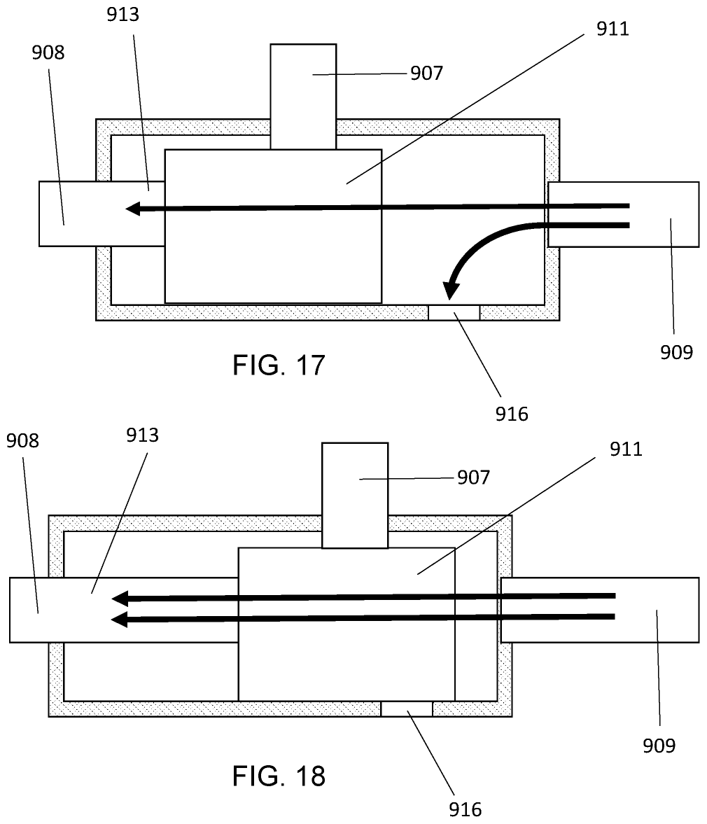

Modulation of the gas pressure occurs by selective partial routing of the gas pressure through one or a plurality of first gas egress ports 916 as shown in FIG. 13. This principle operates similar to that of a musical flute. As shown in FIGS. 17 and 18, selective configuration of the relative positioning of the longitudinally movable tube to block or unblock the first gas egress ports can divert gas flow out of the handpiece through the gas egress ports instead of entirely through the modulated exit gas tube 913. By this manner, gas pressure presented to the modulated exit gas tube 913 is reduced.

In some embodiments, the gas pressure modulator handpiece can further comprise a guard membrane 906 to prevent particles from entering the gas flow stream via the first gas egress ports which would then be transported to the eye. The guard membrane can comprise one or a plurality of second gas egress ports 917 which allow for the diverted gas through the first gas egress ports to escape to atmosphere. The position of the second gas egress ports can be displaced relative to the first gas egress ports such that should a particle enter a second gas egress port, it would not fall into the gas path which is exiting through the modulated exit gas tube. In some embodiments, the total cross-sectional area of the second gas egress ports is larger than the total cross-sectional area of the first gas egress ports so as to not build up a back-pressure within the guard membrane.

In some embodiments, gas pressure can be modulated in the gas modulator handpiece by diverting a partial flow of the gas presented to the gas modulator handpiece by mechanical action within the handpiece. In some embodiments, the partial flow can be performed by the flute-like mechanism described here. In some embodiments, the partial flow can be performed by an escape hatch, such as a partial wall of the outer handpiece wall selectively opening such as by a hingably movable door, or a sliding door along the outer handpiece wall.

### Medical Grade Gas

In some embodiments, the gas is selected from air or medical grade gas. The medical grade gas is selected from SF6 (sulfur hexaflouride), Nitrogen, Argon, Helium, a perfluorohydrocarbon gas, or combinations thereof. The perfluorohydrocarbon gas is selected from: C3F8 (Perfluoropropane), Hexafluoroethane (C2F6), or Perfluoroperhydrophenanthrene. In some embodiments, the gas is filtered before introduction to the subject.

In some embodiments, the concentration of the medical gas can be reduced from neat before presentation to the subject. In some embodiments, the reduced concentration of medical gas can range from 10-14% (v/v) of the medical gas in air or another medical grade gas. In some embodiments, step (d) is performed after step (c).

### Methods of Disrupting or Displace a Sub-retinal Hemorrhage

In some embodiments, this disclosure relates to a method of disrupting or displacing a sub-retinal hemorrhage using the air cannulas and handpieces described herein.

In some embodiments the method of disrupting and/or dispersing a sub-retinal hemorrhage in the eye of a subject in need thereof comprises the steps of:
a. configuring a gas cannula of this disclosure to be proximate to a sub-retinal hemorrhage in the eye of a subject having said subretinal hemorrhage;
b. presenting a pressurized gas to the sub-retinal hemorrhage through the gas cannula; and
c. moving the gas cannula in a transretinal manner to disrupt or disperse the sub-retinal hemorrhage.

In some embodiments, the gas cannula is part of the handpieces described herein.

In some embodiments, moving the gas cannula in a manner to disperse the sub-retinal hemorrhage can be performed by moving the gas cannula in a transretinal manner. In some embodiments, the gas cannula can be moved in a rowed patterned to push the hemorrhage across the retina or away from the retina. In some embodiments, the gas cannula can be moved about orthogonal to the surface of the retina. The purpose of moving the gas cannula is to apply a non-contacting force to the sub-retinal hemorrhage to disperse the blood such that it will be reabsorbed by the body.

In some embodiments, the gas cannula is introduced into the eye through a trocar. The trocar can comprise an internal shape which is about the same shape as the exterior profile of the gas cannula.

In some embodiments, the gas pressure applied to the sub-retinal hemorrhage of the eye of a subject is from 0.1 to 30 psi (pounds per square inch) (5.17 to 1550 mm Hg). In some embodiments, the applied gas pressure is from 1 to 20 psi (51.7 to 1000 mm Hg). In some embodiments, the applied gas pressure is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 psi or any pressure between the aforementioned values.

In some embodiments, the pressurized gas is presented in a continuous flow mode. In some embodiments, the pressurized gas is presented in a pulsed mode. The pulse can be in a square wave or sinusoidal manner. The intervals between the pulses can be between every about 0.1 to about 5 seconds. In some embodiments, the pulse intervals can be between from about 0.1 to about 2 seconds. In some embodiments, the pulse intervals (or peak maxima for sinusoidal pulses) can be between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5 seconds or any time interval between any of the aforementioned times.

In some embodiments, the method further comprises step (d) injecting a reduced concentration of a medical gas (which can include or exclude: nitrogen, SF₆, C₃F_{8 ,} or filtered air) into the eye of the subject, followed by configuring the subject to be in a face down head position. In some embodiments, the subject remains in a face down head position for a period of 1, 2, 3, 4, 5, 6, 7, 8, 9, 10, 11, 12, 13, or 14 days. In some embodiments, the subject remains in a face down head position for 7 days. In some embodiments, the subject is configured to be placed in a face down head position during the steps of presenting a gas cannula to the subject and moving the gas cannula in a transretinal manner to the sub-retinal hemorrhage. In some embodiments, the subject is configured to be placed in a face down head position immediately after performing the steps of presenting a gas cannula to the subject and moving the gas cannula in a transretinal manner to the sub-retinal hemorrhage. In some embodiments, the subject is configured to be placed in a head down position within 30 seconds, 1 minute, 2 minutes, 3 minutes, 4 minutes, 5 minutes, 6 minutes, 7 minutes, 8 minutes, 9 minutes, 10 minutes, 15 minutes, or 30 minutes after performing the steps of presenting a gas cannula to the subject and moving the gas cannula in a transretinal manner to the sub-retinal hemorrhage.

In some embodiments, the method further comprises step (e) injecting an anti-VEGF (anti-vascular endothelial growth factor) agent into the eye of a subject. In some embodiments, step (e) is performed after step (c). In some embodiments, the anti-VEGF agent is selected from: aflibercept (Eyelea™) bevacizumab (Avastin™), ranibizumab (Lucentis™), and combinations thereof.

### Surgical Systems

In some embodiments, this disclosure provides for a surgical system, comprising: a gas cannula of this disclosure; and a handpiece, and a pressurized gas source. In some embodiments, the handpiece is configured to selectively control the fluidic contact of pressured air or medical gas into the open lumen of the gas cannula from the pressured gas source via a valve. In some embodiments, the handpiece is fluidically connected to the gas cannula.

In some embodiments, the surgical system further comprises a gas source controller connected to the proximal end of the gas cannula. The gas source controller can deliver pressured gas to the handpiece such that the operator (e.g., surgeon) can independently control the gas flow rate with a foot vis a foot pedal, whether to apply pressure with a thumb or finger trigger, and the location of the gas cannula to be proximate a sub-retinal hemorrhage by manual manipulation.

In some embodiments, handpiece comprises a toggle switch which controls in a binary fashion the flow of gas through the lumen of the handpiece. In some embodiments, the toggle switch is connected to a lever arm which pushes against a compressible tubing within the handpiece through which the pressurized gas flows.

In some embodiments, the handpiece can further comprises a pressure sensor to measure the gas pressure applied to the gas cannula. In some embodiments, the gas pressure sensor is a MEMS sensor (e.g. IntraSense Miniature Invasive Pressure Sensor, (TE Connectivity)). In some embodiments, the gas pressure sensor can be configured to be positioned at the proximal end of the gas cannula.

In some embodiments, the surgical system can further comprise a foot pedal. The foot pedal can control the gas pressure in a linear fashion as a function of the amount of pressure applied to the pedal or distance of pedal compression.

In some embodiments, the surgical system further comprises a gas controller unit. The gas controller unit can further comprise an air compressor, and/or a filter to deliver sterile gas. In some embodiments, the gas controller unit can be pneumatically connected in a valvable manner to an external gas cylinder tank comprising air or medical grade gas. In some embodiments, the gas controller unit comprises a computer system which comprises a computer monitor and a dial or haptic feedback display which is configured to control the gas pressure.

In some embodiments, the gas controller unit is configured to deliver pressured gas to the handpiece and/or gas cannula at a selected gas pressure (e.g., 0.1 to 20 psi). In some embodiments, the gas pressure is from 1 to 100 mm Hg.

### EXAMPLES

### Example 1

### Embodiment of Air Knife can Disrupt Created Sub-retinal Hemorrhage

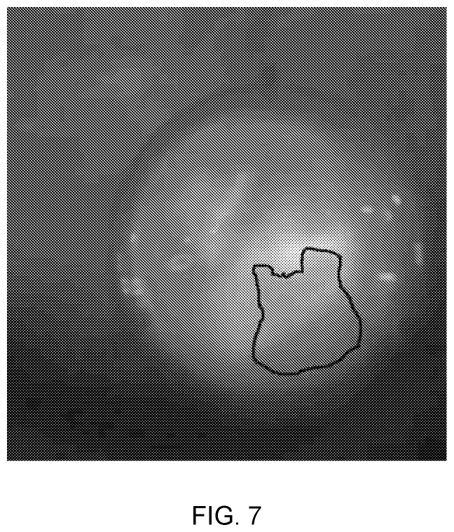

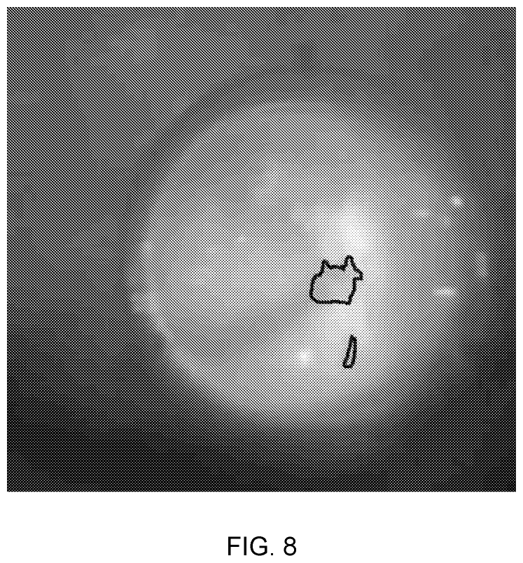

A sub-retinal hemorrhage was created in the sub-retinal space of a rabbit eye in an intraocular surgery of a rabbit eye after pars plana vitrectomy and air fluid exchange to create the sub-retinal hemorrhage. A sub-retinal and pre-retinal blood clot was created by applying continuous airflow to the retina and choroid with a soft tipped canula. A round cannula of this disclosure which produced filtered air with a pressure of 10 mm Hg was used to displace the pre- and sub-retinal clot in a transretinal fashion. FIG. 7 shows a microscopy image of the eyeball with the blood clot outlined. FIG. 8 shows a microscopy image of the eyeball after performing this procedure, where the outline of the blood clot is significantly reduced in area. The experiment demonstrates the successful clearance of the vast majority of the blood clot types (hemorrhage) using a representative embodiment gas cannula of this disclosure.

Although the foregoing specification and examples fully disclose and enable certain embodiments, they are not intended to limit the scope, which is defined by the claims appended hereto.

All publications, patents and patent applications are incorporated herein by reference. While in the foregoing specification certain embodiments have been described, and many details have been set forth for purposes of illustration, it will be apparent to those skilled in the art that additional embodiments and certain details described herein may be varied considerably without departing from basic principles.

The use of the terms “a” and “an” and “the” and similar referents are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the technology and does not pose a limitation on the scope of the technology unless otherwise claimed. No language in the specification should be construed as indicating any non-claimed element as essential to the practice of the technology.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the embodiment.

Embodiments are described herein, including the best mode known to the inventors. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this technology includes all modifications and equivalents of the subject matter recited in the claims appended hereto as permitted by applicable law. Moreover, any combination of the above-described elements in all possible variations thereof is encompassed by embodiments unless otherwise indicated herein or otherwise clearly contradicted by context.

### REFERENCES

All references cited herein are herein incorporated in their entirety.

US Patent No. 9,795,452, Treatment apparatus for a sub-retinal injection and method for assisting in a sub-retinal injection

Heriot W J Intravitreal gas and tPA: an outpatient procedure for submacular hemorrhage. Paper presented at: American Academy of Ophthalmology Annual Vitreoretinal, Update October 1996 Chicago, IL.

Ohji M, Saito Y, Hayashi A, Lewis J M, Tano Y. Pneumatic displacement of sub-retinal hemorrhage without tissue plasminogen activator. Arch Ophthalmol. 1998 Oct;116(10):1326-32.

Hassan A S, Johnson M W, Schneiderman T E, Regillo C D, Tornambe P E, Poliner L S, Blodi B A, Elner S G. Management of submacular hemorrhage with intravitreous tissue plasminogen activator injection and pneumatic displacement. Ophthalmology. 1999 Oct;106(10):1900-6; discussion 1906-7.

Handwerger B A, Blodi B A, Chandra S R, Olsen T W, Stevens T S. Treatment of submacular hemorrhage with low-dose intravitreal tissue plasminogen activator injection and pneumatic displacement. Arch Ophthalmol. 2001 Jan;119(1):28-32.

Holland D, Wiechens B. Intravitreal r-TPA and gas injection in traumatic submacular hemorrhage. Ophthalmologica. 2004 Jan-Feb;218(1):64-9.

Wu T T, Sheu S J. Intravitreal tissue plasminogen activator and pneumatic displacement of submacular hemorrhage secondary to retinal artery macroaneurysm. J Ocul Pharmacol Ther. 2005 Feb;21(1):62-7.

Yang P M, Kuo H K, Kao M L, Chen Y J, Tsai H H. Pneumatic displacement of a dense submacular hemorrhage with or without tissue plasminogen activator. Chang Gung Med J. 2005 Dec;28(12):852-9

Gopalakrishan M, Giridhar A, Bhat S, Saikumar S J, Elias A, N S. Pneumatic displacement of submacular hemorrhage: safety, efficacy, and patient selection. Retina. 2007 Mar;27(3):329-34.

Ron Y, Ehrlich R, Axer-Siegel R, Rosenblatt I, Weinberger D. Pneumatic displacement of submacular hemorrhage due to age-related macular degeneration. Ophthalmologica. 2007;221(1):57-61.

Fang I M, Lin Y C, Yang C H, Yang C M, Chen M S. Effects of intravitreal gas with or without tissue plasminogen activator on submacular haemorrhage in age-related macular degeneration. Eye (Lond). 2009 Feb;23(2):397-406. Epub 2007 Nov 2.

What is claimed is:

1. A gas pressure modulator handpiece comprising:
a. a wall surrounding an open lumen;
b. a longitudinal axis along the open lumen;
c. a proximal end; and
d. distal end,
wherein the distal end comprises one or more openings configured to allow for pressurized air or medical grade gas to propagate and be ejected through the distal end,
wherein the proximal end is in fluidic communication with a filtered gas feed tube,
and wherein the one or more openings comprise a profile configured to propagate and eject dispensed air from said distal end in a laminar flow pattern.

2. The gas pressure modulator handpiece of claim 1, wherein the laminar flow pattern is an air knife.

3. The gas pressure modulator handpiece of claim 1, wherein the open lumen has a longitudinal diameter ranging from 14-31 gauge.

4. The gas pressure modulator handpiece of claim 1, wherein the propagated and ejected gas is propagated in a continuous or pulsed manner.

5. The gas pressure modulator handpiece of claim 1, wherein the one or more distal openings independently comprise a cross-sectional profile selected from a spatulated, fan-shaped, round, prolate round, or rectangular shape.

6. The gas pressure modulator handpiece of claim 5, wherein the one or more distal openings comprise a prolate round shape.

7. The gas pressure modulator handpiece of claim 1, further comprising an inline gas filter, wherein the filtered gas feed tube is in fluidic communication with an inline gas filter.

8. The gas pressure modulator handpiece of claim 7, further comprising an inline gas filter, wherein the inline gas filter is in fluidic communication with a pre-filtered gas feed tube.

9. The gas pressure modulator handpiece of claim 8, further comprising a gas regulator, wherein the pre-filtered gas feed tube is in fluidic communication with a gas regulator.

10. The gas pressure modulator handpiece of claim 9, further comprising a pressurized gas source wherein the gas regulator is in fluidic communication with a pressurized gas source.

11. The gas pressure modulator handpiece of claim 10, further comprising a gas cylinder wherein the pressurized gas source is a pressurized gas cylinder.

12. The gas pressure modulator handpiece of claim 1, wherein the wall surrounding an open lumen comprises one or a plurality of first gas egress ports.

13. The gas pressure modulator handpiece of claim 12, further comprising a slide rack comprising a slider button, a longitudinally moveable tube, and a modulated exit gas tube, wherein the slider button and slide rack are configured to longitudinally move to selectively cover the one or a plurality of first gas egress ports.

14. The gas pressure modulator handpiece of claim 13, further comprising a guard membrane, wherein the guard membrane comprises one or a plurality of second gas egress ports.

15. The gas pressure modulator handpiece of claim 13, wherein the pressure of air directed through the modulated exit gas tube when pressurized air is presented to the gas pressure modular handpiece through the proximal end decreases when the slider button and slide rack are not covering the one or a plurality of first gas egress ports, relative to when the slider button and slide rack are covering the one or a plurality of first gas egress ports.

16. The gas pressure modulator handpiece of claim 13, where the modulated exit gas tube comprises a longitudinal axis which is straight.

17. The gas pressure modulator handpiece of claim 13, where the modulated exit gas tube comprises a longitudinal axis which is curved.

18. The gas pressure modulator handpiece of claim 13, where the modulated exit gas tube comprises two or more longitudinal axes such that the modulated exit gas tube is bent.

19. A surgical system, comprising:
a. the gas pressure modulator handpiece of any of claims 1-8;
b. a gas regulator; and
c. a pressurized gas cylinder,
wherein the handpiece is configured to selectively control the flow rate of the pressurized gas into the modulated exit gas tube.

20. The surgical system of claim 19, wherein the flow rate of the pressurized gas ranges from 0.1 to 20 standard liters per minute (SLPM).

21. The surgical system of claim 19, wherein the pressure of the pressurized gas ranges from 0.1 to 30 psi.

22. The surgical system of claim 19, wherein the pressurized gas is selected from medical-grade gas or air.

23. A method of disrupting and/or dispersing a sub-retinal hemorrhage in the eye of a subject in need thereof, the method comprising:
a. configuring the distal end of a gas pressure modulator handpiece of any of claims 12-18 to be proximate to a sub-retinal hemorrhage in the eye of a subject;
b. presenting a pressurized gas to the sub-retinal hemorrhage through the gas pressure modulator handpiece; and
c. moving the distal end of said gas pressure modulator handpiece in a transretinal manner to disrupt or disperse the sub-retinal hemorrhage.

24. The method of claim 23, wherein the gas cannula is introduced into the eye through a trocar.

25. A kit comprising:
a. the gas pressure modulator handpiece of any of claims 1-18; and
b. a therapeutic agent.

26. The kit of claim 25, wherein the therapeutic agent is selected from an anti-VEGF agent or a steroid.

27. The kit of claim 26, wherein the anti-VEGF agent is selected from: Avastin (bevacizumab), Lucentis (ranibizumab), Eylea (aflibercept), Beovu (brolucizumab), and Vabysmo (faricimab).

28. The kit of claim 27, wherein the steroid is selected from: Triesence (triamcinolone acetonide), and Kenalog (triamcinolone acetonide).
